(19) United States
(12) Patent Application Publication
(10) Pub. No.: US 2003/0133920 A1
(43) Pub. Date: Jul. 17, 2003

(54) Koji molds for preparing cholesterol lowering products

(76) Inventors: Hassan Hajjaj, Fontaines St. Martin (FR); Peter Van Den Broek, Wjmaarn (NL); Peter Niederberger, Epalinges (CH); Laurent-Bernard Fay, Evian (FR); Catherine Mace, Lutry (CH); Jean-Richard Neeser, Savigny (CH)

Correspondence Address: WINSTON & STRAWN, PATENT DEPARTMENT, 1400 L STREET, N.W., WASHINGTON, DC, 20005-3502, US

(21) Appl. No.: 10/350,227

(22) Filed: Jan. 24, 2003

Related U.S. Application Data
(63) Continuation of application No. PCT/EP01/07908, filed on Jul. 10, 2001.

(30) Foreign Application Priority Data
Jul. 28, 2000 (EP) 00116451.6

(51) Int. Cl. A61K 48/00; C12P 7/62; C12N 1/16

(52) U.S. Cl. 424/93.21; 435/254.3; 435/135

(57) ABSTRACT

An isolated, naturally occurring microorganism which is incapable of producing a toxin and which produces at least one compound which can be ingested by a human to lower serum cholesterol concentrations. The microorganism can be used to prepare an edible product such as a fermented food product or a medicament that includes the at least one compound and that can be safely ingested by the human to reduce serum cholesterol concentrations.

### CROSS-REFERENCE TO RELATED APPLICATIONS

This application is a continuation of the US national phase designation of International application PCT/EP01/07908 filed Jul. 10, 2001, the content of which is expressly incorporated herein by reference thereto.

### BACKGROUND

The present invention relates to microorganisms which produce cholesterol lowering compounds and the use of these microorganisms in the production of fermented food products such as bio-hydrolysates, soya sauces or seasonings, for example with cholesterol lowering properties.

Biohydrolysates like soya sauces are traditionally produced through a two or three steps process that include a first step of koji production. In this first step, cooked soya beans or defatted soya flour are mixed with roasted wheat, the mixture is inoculated with a culture of koji mold, cultured under aerobiosis and stirred intermittently for one to four days to give a koji. A second step includes the preparation of a moromi by addition of water and salt to initiate hydrolysis. This moromi is left to ferment with moromi yeasts for about 6 to 8 months. A final step relates to the isolation of the liquid sauce from the solids.

In the context of the invention, the term “koji” designates the product of fermentation, with a koji culture, of a mixture of a protein source and a carbohydrate source, more particularly a mixture of a cooked pulse or oilseed and a cooked or roasted cereal, such as for example, a mixture of cooked soya or haricot bean and cooked or roasted wheat or rice.

In the context of the invention, a koji culture is understood to be a culture or koji spores of the type available on the market, which in particular comprises spores of “yellow Aspergilli”.

Other kind of biohydrolysates can be made by fermenting any other source of protein. Thus materials, preferentially containing glutamic acid-rich proteins, such as for example oilseed cakes, pulses or cereal gluten, are widely used in hydrolyzed form as a starting material in the composition of dehydrated or liquid soups, sauces and seasonings.

The kinds of biohydrolysates that can be used to prepare liquid seasoning are very appreciated because they show strong and rich aromatic taste and are therefore used as seasoning in various meals. These hydrolysates can also be useful as aromatic bases for food or flavoring industries because of their prominent aromatic profile.

Nowadays the nutritional awareness leads the food industry to provide foodstuffs not only with optimal organoleptic features but also with improved nutritional functionalities. The importance of excessive serum cholesterol levels as a main risk factor for the occurrence of atherosclerosis of the coronary and peripheral vasculature is generally recognized. Extensive clinical studies have led to the conclusion that the risk of suffering from coronary diseases can be decreased by reduction of the serum cholesterol level, and in particular of “Low density lipoprotein” levels. Since the major proportion of the cholesterol in the body is derived via de-novo biosysnthesis in the liver and only a smaller part is usually derived by absorption from the food, the inhibition of cholesterol-biosynthesis represents a particularly attractive method of lowering increased plasma cholesterol levels.

In Asian countries for example, several fermented products are used for their beneficial health properties. These food products are often derived from molds fermenting plant raw materials. A good example is Chinese “Red-Yeast-Rice”, a rice fermented with a mold of the genus “Monascus”. This product contains metabolites of the class of “Monacolins” that have been demonstrated to have a cholesterol-lowering effect. These Monacolins belonging to the “statin family” of compounds that act as inhibitors of an enzyme in cholesterol biosysnthesis called “HMGCoA reductase” which in turn is responsible of the conversion of HMGCoA to mevalonate. Statins are successfully applied, in pure form, in pharmaceutical products with the main action to lower LDL-cholesterol level (Low Density Lipoprotein-cholesterol). As a matter of fact, HMGCoA reductase acts as a key enzyme in the biosynthesis of the cholesterol. Thus, the product MEVACOR® from MERCK, for example, contains isolated and purified lovastatin from the filamentous mold Aspergillus terreus. This compound is also related to the statin family and shows inhibitory activity against HMGCoA reductase. On the other hand, PHARMANEX brought a “dietary supplement” called CHOLESTIN® on the market, and it contains molecules of the Monacolin family, and in which the active metabolites are produced by the filamentous mold Monascus purpureus.

However, it has been shown that Monascus purpureus is potentially able to produce a nephrotoxic and/or mutagenic compound called Citrinin (Sabater-Vilar M, Maas R F and Fink-Gremmels J. Mutat. Res. Jul. 21, 1999; 444(1):7-16). For this reason the status of the dietary supplement CHOLESTINE® and, all the more of any potential food products, containing this mold are under strong debate in the USA and in European countries from a legal, safety and toxicological point of view. The same problem may arise with Aspergillus terreus that is known to produce Citrinin.

Therefore Aspergillus terreus or Monascus pupureus are not microorganisms which are applicable in food processes or directly in food products. Thus it is known that several filamentous molds can produce compounds belonging to the statin family, but such molds are not “food-grade” because of potential toxin production and for this reason cannot be used in food fermentation processes and related food products.

EP 556699 (NOVOPHARM) describes the conversion of non-lovastatin producing Aspergillus strains (Aspergillus oryzae) to producing strains by introduction of Aspergillus terreus genomic DNA. This patent teaches the use of genetic modification as a means of obtaining statin-producing filamentous molds, which have a “food-grade” status. The filamentous molds of the claimed species Aspergillus oryzae are therefore not of “natural” but of recombinant origin. However, it would be more desirable to have a natural product of this type.

### SUMMARY OF THE INVENTION

The invention now provides an isolated, naturally occurring microorganism which is incapable of producing a toxin and which produces at least one compound which can be ingested by a human to lower serum cholesterol concentrations.

This microorganism can be used as a food fermenting additive, in that it can be used to ferment a food product to produce the at least one compound but not a toxin, so that the fermented food product can be safely ingested by a human to lower serum cholesterol concentrations.

The invention also relates to a method for preparing an edible composition for lowering serum cholesterol concentrations, which comprises selecting a naturally occurring microorganism which is incapable of producing a toxin and which produces at least one compound which can be ingested by a human to lower serum cholesterol concentrations; and fermenting the microorganism to prepare an edible composition that contains the at least one compound but does not contain a toxin, so that the edible product can be safely ingested by a human to lower serum cholesterol concentrations.

The edible composition can be provided in the form of a medicament for modulation, prevention or treatment of serum cholesterol levels. Alternatively, the edible composition can be provided in the form of a fermented food product prepared by inoculating crop material with an inoculate containing at least one selected microorganism to effect fermentation; and hydrolysing the resultant preparation to obtain a hydrolysate that contains the at least one compound.

The edible products, i.e., the medicament and fermented food products or protein hydrolysates prepared by these methods represent additional embodiments of the invention.

The invention can also relates to a method for lowering serum cholesterol concentrations in a human, which comprises preparing an edible composition by one of the methods disclosed herein; and administering the edible composition to the human in an amount sufficient to lower serum cholesterol concentrations. The edible composition generally contains the at least one compound in an amount which is sufficient to inhibit cholesterol biosynthesis in the human or to inhibit the production of HMGCoA reductase enzyme. The edible composition can also be administered to inhibit at least one step downstream of mevalonate.

### BRIEF DESCRIPTION OF THE DRAWING FIGURES

FIG. 1 is a mass spectrometry (“MS”) graph obtained with lovastatin standard.

FIGS. 2 and 3 show the MS graphs of the extracts obtained by fermentation with microorganisms according to the invention (with strains A-27 and A-4, CNCM I-2489 and CNCM I-2490, respectively).

### DETAILED DESCRIPTION OF THE PREFERRED EMBODIMENTS

The present invention now provides natural or naturally occurring food-grade microorganisms for applications in fermented food that provides cholesterol-lowering properties.

To this end, the microorganism according the invention is an isolated, naturally occurring microorganism which is incapable of producing a toxin and which produces at least one compound which is capable of lowering serum cholesterol concentrations.

The invention also provides a fermented food product that includes this microorganism so that the food product can be consumed or ingested by a person to lower their serum cholesterol concentrations.

The present invention more specifically relates to a process for the production of a fermented food, such as a protein hydrolysate, which comprises the steps of inoculating protein containing material with a microorganism according to the invention to effect koji, adding water to the resulting preparation, and hydrolysing the resulting preparation to obtain the hydrolysate.

Finally, the present invention also concerns the use in a fermented food product of at least one compound stemming from a microorganism according to the invention in the manufacture of a food product or medicament for modulation, prevention or treatment of serum cholesterol levels.

In addition, the invention provides a food product with serum cholesterol-lowering properties characterized in that it contains at least one compound stemming from a microorganism according to the invention.

In the present specification, the term “natural or naturally occurring microorganism” is understood to mean a microorganism that has not been genetically modified and can be selected and isolated from nature i.e., the environment.

Also, the term “incapable of producing a toxin” is understood to mean that the microorganism doe not have the ability to and cannot produce a toxin despite whatever the environmental and/or growth conditions may be.

Furthermore, the term “fermented food product” is understood to mean an edible product whose production process comprises at least one fermentation step involving the use of at least one microorganism according to the invention. Furthermore, the term “edible product” is understood to mean a product whose consumption does not have toxic nor harmful secondary effects on human health. In particular, due to the presence of cholesterol-lowering compounds, such “fermented food products” can provide health-beneficial functionalities. Indeed, the microorganism according to the invention can be selected among microorganisms that exhibit safe-history of use for human consumption.

The cholesterol lowering effect obtained by the use of a microorganism according to the invention is achieved by inhibiting cholesterol biosynthesis. In a particular embodiment of the invention, the cholesterol lowering effect is achieved by inhibition of HMGCoA reductase. Indeed, one of the cholesterol-lowering compounds produced by a microorganism according the invention which is capable of lowering cholesterol concentrations through HMGCoA reductase inhibition, is lovastatin.

However, a microorganism according to the invention can produce at least one cholesterol-lowering compound that is not lovastatin and that can also inhibit cholesterol biosynthesis. HMGCoA reductase catalyses the conversion of HMGCoA to mevalonate, but this step is just one step is the whole cholesterol biosynthesis chain. At least one of the compounds produced by a microorganism according to the invention inhibits at least one step downstream of mevalonate. Such downstream inhibition can be observed by adding mevalonate to shunt the HMGCoA/Mevalonate step.

One of the key features of the microorganism according to the invention is that it is incapable of producing a toxin, particularly incapable of producing an aflatoxin. The microorganism that is capable of producing at least one cholesterol-lowering compound without any production of toxin may be a microorganism used in the preparation of the product, for example, particularly a filamentous mold such as Monascus, Penicillium and more preferentially molds belonging to the genus Aspergillus, for example, providing that the microorganism be selected for its ability to synthesize cholesterol-lowering compounds but its inability to produce a toxin. Then, this microorganism maybe selected among the broad variety of microorganisms having a safe history of use in human consumption and usable for food production or in a food product itself.

In a preferred embodiment of the present process, the food product made through the use of a microorganism according to the present invention may be a liquid seasoning, such as a soya sauce, or a seasoning paste or powder, for example.

The microorganism according to the invention can be reduced to practice in a traditional soya sauce or seasoning production process. It can also be used, alone or in combination with other koji molds in any process such as the ones described in EP 0429760 (process for preparing a flavoring agent), EP 0829205 (seasoning product) or EP 0824873 (production of a seasoning product).

The invention also uses one or more of these microorganisms in the manufacture of a fermented food product or medicament for modulation, prevention or treatment of serum cholesterol levels. Alternatively, the invention uses at least one compound stemming from a microorganism according to the present invention in the manufacture of a food product or medicament for modulation, prevention or treatment of serum cholesterol levels.

Among the different steps of the process of the invention, a characteristic one is the fermentation of a plant material with at least one microorganism according to the invention, for example, or with a combination of at least one microorganism according to the invention and a traditional fermenting microorganism that does not produce cholesterol-lowering compound. For example, this first koji step may also be conducted in the presence of added proteolytic enzymes from various origins.

In a preferred embodiment where the process is related to the manufacturing of a liquid seasoning such as soya sauce, the microorganism is a mold, preferentially a koji mold such as one belonging to the genus Aspergillus, that has been selectively selected and isolated among the wide variety of existing koji mold strains for its ability to produce at least one cholesterol-lowering compound but its lack of a potential to produce toxin. To this end and for this preferred embodiment, 12 strains of the genus Aspergillus have been deposited, by way of example, according Budapest Treaty, on June 14, 2000 and on Jul. 27, 2000 at CNCM, Collection Nationale de Culture de Microorganismes, Institut Pasteur, Rue du Dr Roux, 75724 Paris, France, where they received the identifications numbers CNCM 1-2489, CNCM 1-2490, CNCM 1-2525, CNCM 1-2526, CNCM 1-2527, CNCM 1-2528, CNCM 1-2529, CNCM 1-2530, CNCM 1-2531, CNCM 1-2532, CNCM 1-2533 and CNCM 1-2533.

In such a preferred embodiment, the protein containing material may be a plant material such as wheat grains, soya, rice, or maize, oil, seeds, or fractions of plant materials such as wheat gluten or wheat bran, or any mixture of such plant materials or plant material fractions. The different steps of the process of soya sauce production can be done according to traditional know-how of the skilled artisan of the manufacturing of fermented sauces such as soya sauces. The main feature is the use or the addition of microorganisms according to the invention, and preferentially the addition of such microorganisms to koji molds.

The soya material may be cooked, for example, by soaking crushed soya beans or soya meal for a few hours and then subjecting the meal to a temperature of approximately 120° C. to 140° C. for a few minutes. If desired, the cooked soya meal may be mixed with other plant materials, such as crushed roasted grains of wheat, i.e., a roasted wheat meal.

After cooling, the mixture may be inoculated with spores of the koji mold according to the invention or a mix of these mold spores and traditional ones from commercial suppliers. The mixture may then be left to ferment on a tray or in a commercial apparatus that is specially designed for this purpose with intermittent stirring and constant aeration. This fermentation step promotes the production of proteases by the koji molds present in the mix and in particular at least one compound which is capable of lowering cholesterol concentrations by the molds according to the invention that are present in this mix.

The koji mix is then suspended, such as by mixing with water. Then a hydrolysis step is carried out, such as for several hours at 30° C. to 60° C.

The subsequent steps can be carried out according classical process that is known to skilled artisans. Thus, the moromi step maybe realized by the addition of salt and a traditional moromi yeast of the species Candida versatilis or Saccharomyces rouxii. The moromi thus inoculated maybe left to ferment from days to months with stirring and aeration in a conventional manner.

The moromi may then be pressed, for example in a filter press, the insolubles may be removed and the liquor obtained may be pasteurized.

Accordingly, it is possible by this particular process to obtain a fermented liquid seasoning such as this soya sauce which is comparable in taste and aroma with a fermented soya sauce obtained by a traditional process. The principal feature of this soya sauce, beyond its aroma, is its health beneficial functionality due to the presence of at least one cholesterol lowering compound contained in it but without trace of any toxins, e.g., aflatoxin. The resulting fermented liquid can be used either as a seasoning or as a cholesterol lowering dietary supplement.

The microorganism according to the invention, which can be any food microorganism but notably is used for the preparation of the fermented food product, is capable of producing at least one cholesterol-lowering compound but is lacking the ability to produce a toxin and therefore is usable in the manufacturing of any fermented food-stuffs fabrication. The microorganism according to the invention can be any filamentous molds presently or previously used for production of fermented aromatized products (e.g., sake, soya sauce, vegetable seasoning, peanut sauce, etc.) provided that the microorganism is selected and isolated among all the natural existing microorganisms for its ability to produce at least one cholesterol-lowering compound but no to produce toxins.

Thus, in order to select and identify microorganisms that respond to the previous conditions, different kinds of microorganisms from different sources can be checked and submitted to a selection method. The examples presented below are given as illustrations of selection methods for identifying microorganisms that meet the criteria of the invention. This specific selection method comprises the steps of culturing microorganisms on a growth medium, preparing and purifying the culture crude extracts and submitting the latter to lovastatin and toxin analysis and to a test measuring cholesterol synthesis inhibition. Such a method can be applied to any kind of microorganism in order to check if it satisfies the desired criteria. Furthermore, any other equivalent method for cultivating microorganisms, isolating and detecting lovastatin and/or toxin, evaluating influence of the fermentative extracts on cholesterol biosynthesis can be used in order to identify the microorganisms that fulfil the criteria and provide the necessary features of the microorganism of the invention.

### EXAMPLES

The methods for selecting microorganisms that are described in the following examples are not limited to the specific microorganisms that are screened. The toxin detection and identification method is presently directed to aflatoxins class compounds but one of ordinary skill in the art can modify and adapt these procedures for any toxic compounds.

### Example 1

Microorganism Growth Conditions:

Approximately 10⁷ conidiospores were used to inoculate forty milliliters (ml) of medium A in a 250 ml unbaffled Erlenmeyer flask for primary seed culture. The seed medium (medium A) contained per liter: 10 g of glucose, 5 g of corn steep liquor, 40 g of tomato paste, 10 g of oatmeal, and trace elements: FeSO₄.7H₂O 1 g, MnSO₄.4H₂O 1 g, ZnSO₄.7H₂O 200 mg, CaCl₂.2H₂O 100 mg, CuCl₂.2H₂O 25 mg, H₃BO₃ 56 mg, and (NH₄)₆MO₇O₂₄.4H₂O 19 mg. These cultures were incubated for 24 hours at 30° C. in an orbital shaker at 200 rpm.

Secondary cultures were prepared by adding 6 ml of the primary seed culture to a 1 l unbaffled Erlenmeyer flask containing 200 ml of production medium (B).

The production medium (B) contained per liter: 45 g of glucose or lactose, 24 g of peptonized milk, 2.5 g of yeast extract, and 2.5 g of polyethylene glycol P2000 (pH 6.5). The flasks were shaken in an orbital shaker at 200 rpm at 28° C. for 12 days.

Extraction and Purification:

After fermentation (280 h), the culture (200 ml) was macerated with 80% methanol (200 ml) for 2 hours. The mixture was filtered through a filter paper and the filtrate was evaporated. The crude extract was recovered with water and this solution was acidified and adjusted to pH 3 with 3 N hydrochloric acid (HCl).

The aqueous extract was extracted several times with ethyl acetate (v-v). The organic phase was dried with anhydrous Na₂SO₄ and evaporated in vacuum (30° C.) to remove the organic solvent. The residue was taken up in 10 ml MeOH for analysis chromatography (HPLC and TLC), spectroscopy (LC, MS and LC/MS/MS) and in-vitro test (inhibition of the enzyme 3-hydroxy-3-methylglutaryl-coenzyme A reductase [HMG-COA reductase; mevalonate: NADP⁺ oxidoreductase (CoA-acylating), EC 1.1.1.34]) to identify and quantify lovastatin. The production of aflatoxin was evaluated by HPLC.

Lovastatin Analysis:

Lovastatin was determined by high-performance liquid chromatography (HPLC) and mass spectroscopy.

HPLC ANALYSIS: nucleosil 100-5 C₁₈ column (250×4 mm) (Macherey & Nagel) was used with a post column (Lichrospher 100 RP-18 (Merck). Solvent A: 0.05% H₃PO₄ in water, solvent B was acetonitrile. The separation started with a linear gradient from 95% A and 5 % B, reaching 50% A and 50% B in 45 min, the 30% A and 70% B in 46 min, then 10% A and 90% in 48 min, then 0% A and 100% B in 50 min and continued with an isocratic run for 4 minutes. Initial conditions were maintained for 6 minutes to re-equilibrate the column. The flow rate was 1 ml/min. The detector used was Hewlett Packard G1315 A, series 1100, the wave length detection was at λₘₐₓ=254 nm.

HPLC/MS and HPLC/MS/MS ANALYSIS: Separation was performed using a Waters HPLC system, consisting of a type 757 auto sampler, a 600-MS pump with system controller and a type 486-MS UV-detector. The UV absorption at 258 nm was recorded using an analogue input to the mass spectrometer's data system. A Nucleosil 100-C18 HPLC column (250 mm×4 mm I.D., Macherey & Nagel) was used with a post column splitter 1/10 before the mass spectrometer. Solvent A was 0.1% trifluoroacetic acid in water, solvent B was acetonitrile. Using a flow rate of 1 mL/min the separation started with a linear gradient from 95% A and 5% B, reaching 50% A and 50% B in 30 min, then 30% A and 70% B in 31 min, then 10% A and 90% B in 33 min, then 0% A and 100% B in 35 min and continued with an isocratic run for 5 minutes. Initial conditions were reached within 5 minutes and were maintained for 5 minutes to re-equilibrate the column.

The mass spectrometer was a Finnigan TSQ 700 triple quadrupole mass spectrometer (San Jose, Calif., USA) equipped with an electrospray ionization source. Data acquisition was performed on a DECstation 2100 running under Ultrix 4.2A (Digital Equipment, USA) using the Finnigan software package ICIS2, Version 7.0. The transfer capillary was set at 200° C. and the spray at 4.2 kV. Full scan mass spectra were acquired in positive mode by scanning from m/z 50 to m/z 600 in 1 second. Daughter ion spectra were obtained from m/z 20 to m/z 450 at a collision energy of −15 eV in the laboratory frame using argon at a pressure of 1 mTorr as the collision gas. Lovastatin was detected using the same conditions after selective reaction monitoring of the daughter ions at m/z 199, 285 and m/z 303 from the protonated parent ion at m/z 405. Detection by LC/MS/MS of these closely structure-related daughter ions allows the specific determination of lovastatin in the samples.

Aflatoxin Analysis:

The concentrations of individual aflatoxins in methanolic extracts was measured by isocratic HPLC using postcolumn derivatization with on-line electrochemically-generated bromine and fluorescence detection. A reversed-phase ODS Hypersil column (3 μm, 125 mm×4.6 mm i.d.) was used along with an ODS Hypersil guard column (3 μm, 25 mm×4.6 mm i.d.), both from Metrohm-Bischoff AG (Leonberg, Germany). The mobile phase consisted of a mixture of water/acetonitrile/methanol (60:5:35) v/v/v, containing 119 mg potassium bromide and 100 μl 65% nitric acid per liter. Elution was carried out at a flow rate of 1.0 ml/min and at room temperature. The postcolumn derivatization system consisted of a KOBRA cell with variable control current source (Rhône Diagnostics Technologies Ltd, Glasgow, Scotland). Current setting was adjusted at 100 μA. The individual aflatoxins were monitored with a Waters model 470 scanning fluorescence detector (excitation wavelength 365 nm, emission wavelength 428 nm).

The presence of aflatoxins in positive culture supernatants was confirmed by using a more selective method based on an immunoaffinity column cleanup, according to the procedure described by Trucksess et al. (1991). 1 ml aliquots of methanolic extracts were diluted with 50 ml distilled water and applied to an AflaTest-P immunoaffinity column (Vicam, Watertown, Mass., USA) containing a monoclonal antibody specific for aflatoxins B₁, B₂, G₁, and G₂. The toxins were isolated, purified, and concentrated on the column, removed from antibodies with pure methanol, and quantified by reversed-phase HPLC with postcolumn derivatization and fluorescence detection, under the same conditions as previously described.

Inhibition Test for Cholesterol Synthesis:

The human hepatic T9A4 cells were grown in the serum-free LCM medium (Biofluids, Rockville, Md., USA) under 3.5% CO₂ at 37° C. The cells were seeded into 24 well plates and incubated at confluency with 1 mM ¹⁴C-acetate (1 mCi/mmol, Amersham) for 20 h in the absence (control) or in the presence of the fermented extract fractions. Cells were also incubated with 1.17 mM ¹⁴C-mevalonate (0.85 mCi/mmol, Amersham) for 20 h in order to evaluate whether the compounds stemming from the microorganisms to be tested have an inhibitory effect downstream the HMGCoA/Mevalonate conversion step.

Lipid extraction was performed twice by incubation with hexane:isopropanol (3:2) for 30 min at room temperature. The combined extracts were dried under N₂, redissolved in hexane and subjected to high performance thin layer chromatography (Merck, Darmstadt, Germany) in a solvent mixture of hexane:diethyl ether:acetic acid (75:25:1). The neo-synthesis of cholesterol was determined by measuring the ¹⁴C-acetate incorporation into the cholesterol with an instant imager (Camberra Packard, Zurich, Switzerland) and expressed as percent of the control.

Table 1 shows the in vitro hypocholesterolemic activity in the human hepatic T9A4 cells of the extracts in presence of ¹⁴-C acetate. It can easily be seen that the cholesterol synthesis in human hepatic T9A4 cells is reduced when the extracts obtained by fermentation with a microorganism according to the invention are incubated with the cells. Since the lovastatin content in extract obtained from A4 an A27 strains is less than 0.1 μg/ml, the cholesterol synthesis inhibition effect observed can be attributed to lovastatin present in the extracts but also to other compounds similar to the ones that are produced by the other non-lovastatin producing strains.

The extracts obtained with strains A12, A21, A34, A39, A45, A50, A51, A53, FJ2 and FJ5 do not contain any trace of lovastatin (MS graph not shown) but has seen in Table 1, these extracts exhibit strong cholesterol biosynthesis inhibitory activity despite the lack of lovastatin. This indicates that such microorganisms according to the invention produce compounds other than lovastatin capable of lowering serum cholesterol concentrations that act as inhibitors of at least one step downstream of the step catalysed by HMGCoA reductase.

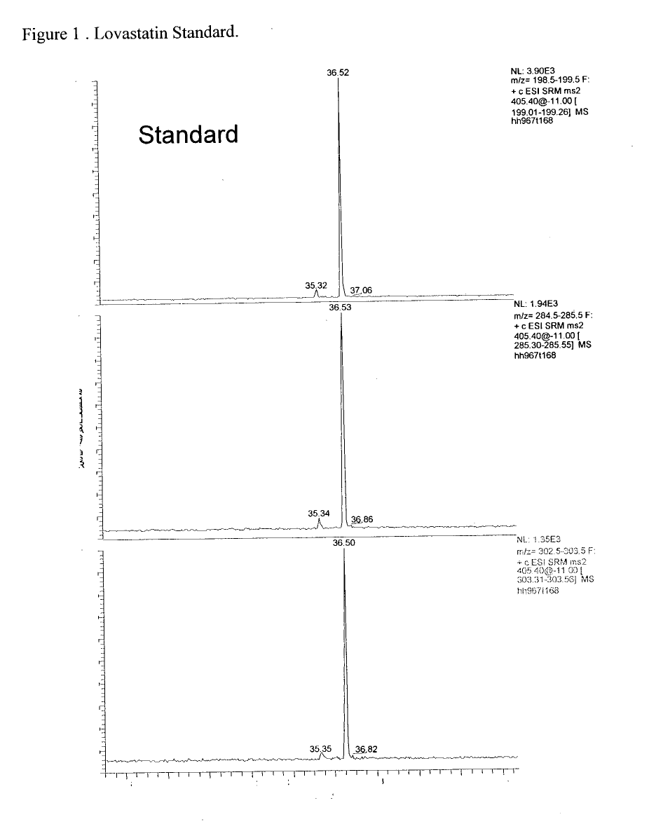

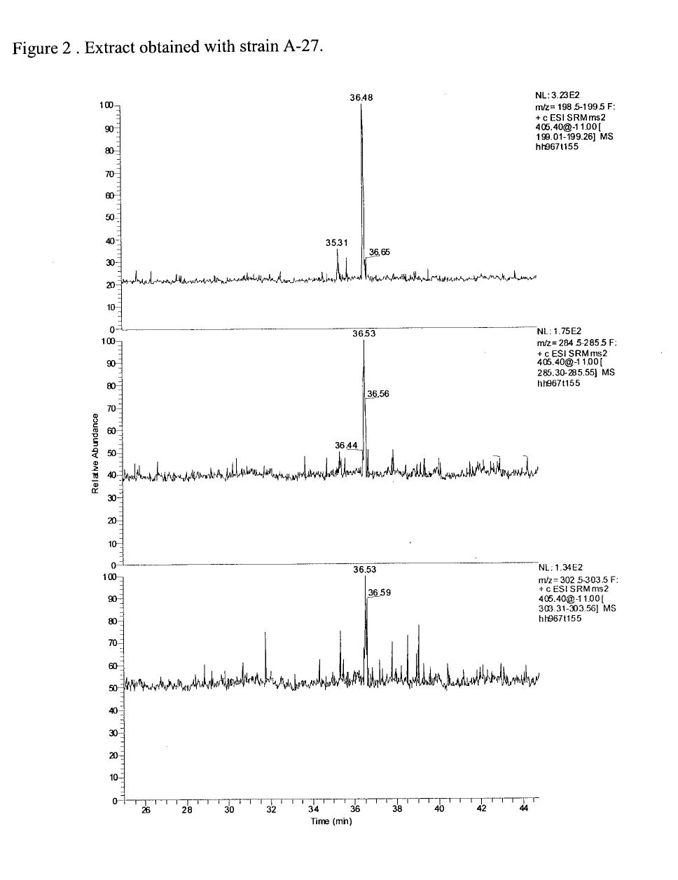

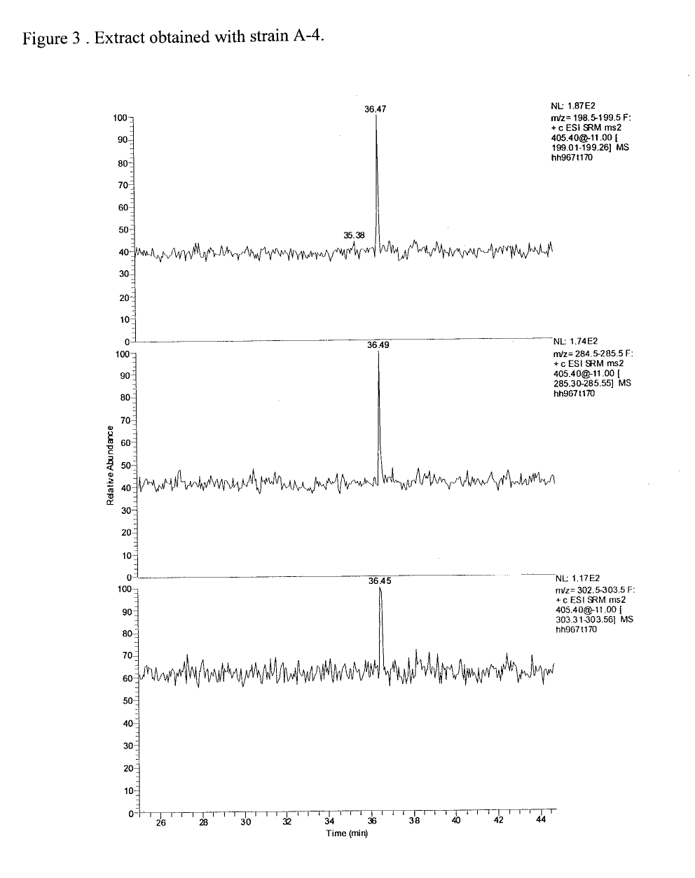

FIG. 1 shows the MS graph obtained with lovastatin standard. FIGS. 2 and 3 show the MS graphs of the extracts obtained by fermentation with microorganisms according to the invention (with strains A-27 and A-4, CNCM 1-2489 and CNCM 1-2490, respectively). lovastatin content of the extracts obtained with these strains is less than 0.1 μg/ml.

Of course none of the extracts obtained with the microorganisms according to the invention contain any trace of aflatoxin.

Table 2 shows the in vitro hypocholesterolemic activity in the human hepatic T9A4 cells of the extracts in presence 14C-mevalonate. In this case, it can be seen that the presence of lovastatin does not inhibit cholesterol biosynthesis even though the fermentation extracts obtained with the strains A21, A34, A39, FJ2 and FJ5 shows evidence of inhibitory effect on cholesterol biosynthesis. This means that these strains produce at least one compound that inhibits at least one step downstream of mevalonate.

TABLE 1
The in vitro hypocholesterolemic activity in human hepatic
assays of strains extracts in presence of ¹⁴C-acetate

| Strain | Extract concentration | % Inhibition of |
|---|---|---|
| code | in the medium (μg/ml) | cholesterol synthesis |
| A4 | 39 | 23 |
| A27 | 51 | 45 |
| A12 | 70 | 59 |
| A21 | 38 | 54 |
| A34 | 50 | 55 |
| A39 | 47 | 64 |
| A45 | 44 | 47 |
| A50 | 43 | 29 |
| A51 | 41 | 7 |
| A53 | 42 | 55 |
| FJ2 | 37 | 61 |
| FJ5 | 40 | 46 |
| LOVASTATIN | 0.5 | 56 |

TABLE 2
The in vitro hypocholesterolemic activity in human hepatic
assays of strains extracts in presence of ¹⁴C-mevalonate

| Strain | Extract concentration | % Inhibition of |
|---|---|---|
| code | in the medium (μg/ml) | cholesterol synthesis |
| A21 | 38 | 82 |
| A34 | 50 | 66 |
| A39 | 47 | 72 |
| FJ2 | 37 | 64 |
| FJ5 | 40 | 42 |
| LOVASTATIN | 0.5 | 0 |

### Example 2

Soy Koji Fermentation

175 g of a mixture of defatted soybeans (75% w/w) and roasted wheat (25% w/w) were steam-cooked for 7 minutes at 120° C. with closed lid in an autoclave and allowed to cool below 40° C. The cooked soybean-wheat mixture was inoculated with 10⁹ cfu of a conidiospore suspension of microorganism according to the invention (strain A27) in SP2 (20 mM KH₂PO₄—HCl pH 2.0, 0.9% NaCl). The inoculated mixture was transferred to a 3-liter mushroom spawn bag (porosity 25 cc/min, filter with 160 mm, Van Leer, United Kingdom) and incubated at 30° C. in a Lab-Term incubator (Kühner, Switzerland) for up to 70 hours. Temperature inside the Koji bed was monitored not to exceed 34° C.

The obtained koji is used for doing an extract according to the method used in Example 1 for obtaining an extract from a liquid fermentate.

It can easily seen from Table 3 that the extracts obtained from solid koji fermentation exhibit cholesterol synthesis inhibition property.

### Example 3

Wheat Bran Koji Fermentation

The method is the same that the one of example 2 except the use of a mix of 120 g Wheat bran (dry matter: 86%)+73 g Water +100 μl Acetic acid, instead of the mix of soya beans and roasted wheat.

The extract is obtained by a method similar to the one described above.

It can easily seen from Table 3 that the extracts obtained from solid koji fermentation exhibit cholesterol synthesis inhibition property.

TABLE 3
The in vitro hypocholesterolemic activity in human hepatic
assays of strains extracts in solid state in presence of ¹⁴C-acetate

| | | % Inhibition of |
|---|---|---|
| Strain Code | Concentration μg/ml | cholesterol synthesis |
| A27 ^{a}(K) | 11 | 45 |
| A27 ^{a}(WB) | 30 | 42 |

^{a}K: Koji medium
^{a}WB: Wheat bran medium

Although this example illustrates the use of wheat bran, the skilled artisan realizes that other crop or plant materials can be substituted for wheat bran depending upon the final product that is desired. Accordingly, the invention can be used to form a wide variety of edible products that can be consumed to lower sserum cholesterol concentrations.

What is claimed is:

1. An isolated, naturally occurring microorganism which is incapable of producing a toxin and which produces at least one compound which can be ingested by a human to lower serum cholesterol concentrations.

2. The microorganism of claim 1 wherein the compound which is capable of lowering serum cholesterol concentrations is lovastatin.

3. The microorganism according to claim 1 wherein the microorganism is incapable of producing an aflatoxin.

4. The microorganism according to claim 1 which is a filamentous mold.

5. The microorganism according to claim 4 wherein the filamentous mold is selected from the group consisting of the genus Aspergillus, Monascus and Penicillium.

6. A food fermentation additive comprising the isolated naturally occurring microorganism of claim 1 which, when used to ferment a food product, produces the at least one compound but does not produce a toxin, so that the fermented food product can be safely ingested by a human to lower serum cholesterol concentrations.

7. A method for preparing an edible composition for lowering serum cholesterol concentrations, which comprises:
selecting a naturally occurring microorganism which is incapable of producing a toxin and which produces at least one compound which can be ingested by a human to lower serum cholesterol concentrations; and
fermenting the microorganism to prepare an edible composition that contains the at least one compound but does not contain a toxin, so that the edible product can be safely ingested by a human to lower serum cholesterol concentrations.

8. The method of claim 7, wherein the edible composition is in the form of a medicament for modulation, prevention or treatment of serum cholesterol levels.

9. A medicament prepared by the method of claim 8.

10. The method of claim 7, wherein the edible composition is in the form of a fermented food product prepared by:
inoculating crop material with an inoculate containing at least one selected microorganism to effect fermentation; and
hydrolysing the resultant preparation to obtain a hydrolysate that contains the at least one compound.

11. The method of claim 10, wherein the crop material is wheat grains, soya, rice, or maize; oil, seeds, or fractions of such crop materials; or a mixture of such crop materials or crop material fractions.

12. A fermented food product prepared by the method of claim 10.

13. A method for lowering serum cholesterol concentrations in a human, which comprises:
preparing an edible composition by the method of claim 7; and
administering the edible composition to the human in an amount sufficient to lower serum cholesterol concentrations.

14. The method of claim 13, wherein the edible composition contains the at least one compound in an amount which is sufficient to inhibit cholesterol biosynthesis in the human.

15. The method of claim 13, wherein the edible composition contains the at least one compound in an amount which is sufficient to inhibit the production of HMGCoA reductase enzyme.

16. The method of claim 13, wherein the edible composition contains the at least one compound in an amount which is sufficient to inhibit at least one step downstream of mevalonate.

17. A method for lowering serum cholesterol concentrations in a human, which comprises administering the medicament of claim 9 in an amount sufficient to lower serum cholesterol concentrations.

18. A method for lowering serum cholesterol concentrations in a human, which comprises administering the fermented food product of claim 12 in an amount sufficient to lower serum cholesterol concentrations.

19. A method for lowering serum cholesterol concentrations in a human, which comprises:
preparing a fermented food product with the food fermentation additive of claim 6; and
administering the fermented food product to the human in an amount sufficient to lower serum cholesterol concentrations.

20. The method of claim 19, wherein the fermented food product comprises a protein hydrolysate.
